(19) United States
(12) Patent Application Publication
(10) Pub. No.: US 2026/0027300 A1
(43) Pub. Date: Jan. 29, 2026

(54) MEDICAL DEVICE AND MEDICAL SYSTEM USING SAID MEDICAL DEVICE

(71) Applicants: Carlo DE ASMUNDIS, Dworp (BE); Giacomo TALEVI, Falconara Marittima (AN) (IT); Alvja MALI, Falconara Marittima (AN) (IT)

(72) Inventors: Carlo DE ASMUNDIS, Dworp (BE); Giacomo TALEVI, Falconara Marittima (AN) (IT); Alvja MALI, Falconara Marittima (AN) (IT)

(21) Appl. No.: 18/998,438

(22) PCT Filed: Jul. 5, 2023
(86) PCT No.: PCT/IB2023/056945

§ 371 Date: Jan. 24, 2025

(30) Foreign Application Priority Data
Jul. 26, 2022 (IT) 102022000015768

(51) Int. Cl. A61M 5/172; A61M 37/00; H01M 8/04313; H01M 8/04537; H01M 8/04746; H01M 8/04858; H01M 8/16; H01M 16/00

(52) U.S. Cl. CPC A61M 5/1723; A61M 37/0015; H01M 8/04313; H01M 8/04552; H01M 8/04582; H01M 8/04611; H01M 8/04641; H01M 8/04753; H01M 8/04947; H01M 8/16; H01M 16/006; A61M 2205/8206; H01M 2220/30; H01M 2250/30; H01M 2250/402

(57) ABSTRACT

A medical device includes a holder that may be applied to the skin of a patient, a biofuel cell, a pump configured for supplying blood to the biofuel cell, a sensor configured to emit a signal representative of a blood parameter, a control unit connected to the pump and the sensor, and a rechargeable battery electrically connected to the biofuel cell. The control unit is configured to perform a recharging procedure including the following steps: processing the signal emitted by the sensor, estimating a value of the blood parameter of the patient, comparing said estimated value with a threshold value, and, based on the comparison, commanding the activation of the pump for delivering a predetermined amount of blood to the biofuel cell for generation electricity and recharging the battery.

### TECHNICAL FIELD

The present invention relates to a medical device and a medical system using said medical device. A method for recharging said medical device and said medical system are further described. The medical device and medical system may be used in the field of medical instrumentation, such as for monitoring and caring of diabetic and cardiac patients.

### BACKGROUND ART

Medical devices for monitoring the state of health of a patient are known in the art; these devices may be either implantable or extracorporeal, namely they may be applied to the patient's skin.

For example, there are known implantable medical devices equipped with a glucose sensor and a wireless transmitter suitable for transmitting data collected by the sensor to an external mobile device, through which the patient himself or trained medical personnel monitor blood glucose levels to prevent hyperglycemic or hypoglycemic conditions.

Implantable medical systems that use biofuel cells to generate electricity are also known; see, for example, patent applications No. US 2012/283968 A1 and No. US 2018/233761.

Although such devices are used today, for example, to monitor a patient, the Applicant noted that they require a highly invasive installation and maintenance procedure for the patient.

Extracorporeal medical devices for glucose monitoring, applied to a patient's skin, are also known; such devices have an external glucose sensor and a wireless transmitter suitable for transmitting data collected by the sensor to a mobile device. Unlike implantable devices, extracorporeal medical devices do not require invasive installation and maintenance procedures. The Applicant noted, however, that even such known medical devices require constant and careful maintenance by the patient; for this reason, known external medical devices are unreliable in that, proper functioning is entirely up to the maintenance performed by the patient himself. In fact, in the event that constant and careful maintenance is not performed by the patient, the medical device may not function properly, thus preventing the proper monitoring of the patient's health status.

An additional extracorporeal medical device is described in U.S. Pat. No. US 9 647 289 B1. Such device includes a container that may be attached to a patient and configured to receive a flow of blood; in the container there is a solution that, when in contact with a patient's blood, allows to lower the patient's blood sugar level.

It is also known from U.S. patent application No. US 2019/260053 A1 a medical device comprising a biofuel cell using the electro-oxidation of blood glucose to produce electricity, which is used to power the medical device itself. In detail, the medical device includes a rechargeable battery that is electrically connected to the biofuel cell and configured to recharge from the energy produced by the biofuel cell. The medical device is also equipped with a pump used to deliver a dose of insulin into the patient upon the occurrence of a hyperglycemic condition or to deliver glucose upon the occurrence of a hypoglycemic condition. Although the solution described in the U.S. patent application reduces the need for battery replacement due to the presence of the biofuel battery, the Applicant noted that the device described in application No. US 2019/260053 A1 is not without drawbacks. In fact, such medical device requires the use of two different reservoirs, one containing insulin and one containing a glucose solution, giving the device a high structural complexity and a large footprint. Additionally, the Applicant noted that the presence of the reservoirs (insulin and glucose) requires more maintenance, a condition that makes the device unsafe.

### OBJECT OF THE INVENTION

The object of the present invention is to solve at least one of the drawbacks and/or limitations of the previous solutions. It is an object of the present invention to provide a non-invasive medical device that is easy to apply, at the same time capable of operating for long periods of time without the need for maintenance by a trained medical professional or by the patient himself. It is then object of the present invention to provide a medical device suitable for performing precise monitoring of parameters in the blood and ensuring optimal health conditions for the patient. It is also object of the present invention to provide a medical device having a simple and compact structure, which has low production costs; in particular, it is object of the present invention to provide a mobile extracorporeal device, easily applicable on a patient and thus easily transportable. It is also an object to provide a simple and compact medical system that minimizes the maintenance of the system itself.

These objects and others, which will appear from the following description, are substantially achieved by a medical device and a medical system according to one or more of the following claims and/or aspects.

### SUMMARY

In one aspect there is a medical device (1) comprising:
a holder (2) applicable on the skin of a patient (P),
at least one rechargeable battery (6) carried by the holder (2),
at least one biofuel cell (3) carried by the holder (2) and connected to the battery (6), wherein the biofuel cell (3) is configured for receiving a fluid from the patient (P) and producing electrical energy for recharging the battery (6),
at least one pump (4) carried by the holder (2) and configured for supplying the fluid of the patient to the biofuel cell (3),
at least one sensor (5) carried by the holder (2) and configured for emitting a signal representative of a control parameter of the fluid of the patient.

In one aspect according to the preceding aspect, the fluid of the patient includes blood and/or interstitial fluid.

In an aspect according to any one of the preceding aspects, the medical device (1) comprises a control unit (50) connected to the pump (4) and the sensor (5) and active in control on said pump (4), wherein the control unit (50) is configured for performing a power generation procedure comprising the following steps:
receiving a signal from the sensor (5),
processing the signal emitted by the sensor (5) and estimating a value of the control parameter of the fluid of the patient (optionally blood and/or interstitial fluid),
comparing the estimated value of the control parameter with a threshold value, and
based on said comparison, commanding activation of the pump (4) for supplying the fluid of the patient (optionally blood and/or interstitial fluid) to the biofuel cell (3) for producing electricity.

In an aspect according to the preceding aspect the control unit (50), based on the comparison between the estimated value of the control parameter and the threshold value, is configured for transmitting a predetermined amount of fluid of the patient (optionally blood and/or interstitial fluid) to the biofuel cell (3). In an aspect according to any one of the preceding aspects, the control parameter of the fluid of the patient (optionally blood and/or interstitial fluid) includes at least one of: blood glucose, glycated hemoglobin, oxygen concentration, heart rate, blood pressure, temperature, and amino acid or fat concentration. In one aspect according to any one of the preceding aspects, the threshold value is representative of at least one of the following parameters: blood glucose, glycated hemoglobin, oxygen concentration, heart rate, blood pressure, temperature, amino acid or fat concentration.

In one aspect according to any one of the preceding aspects the sensor (5) is an optical sensor and is configured for generating an electrical signal representative of the control parameter. In an aspect according to any one of the preceding aspects the sensor (5) is a glucose sensor. In an aspect according to any one of the preceding aspects the sensor (5) comprises at least one selected from the group of: an infrared sensor, a luminescence sensor, an electromagnetic radiation sensor, an acoustic sensor, an ultrasonic sensor, an impedance measurement circuit, a radio frequency sensor.

In one aspect according to any one of the preceding aspects, the control unit (50), during the estimation step of the value of the control parameter of the power generation procedure, is configured for estimating the value of glucose in the fluid of the patient (optionally blood and/or interstitial fluid).

In an aspect according to any one of the preceding aspects the control unit (50), during the comparison step of the power generation procedure, is configured for comparing said glucose value with a threshold value. In an aspect according to the preceding aspect the threshold value is comprised between 60 mg/dl and 100 mg/dl.

In an aspect according to any one of the preceding aspects, the activation step of the pump (4) of the power generation procedure is performed in case the control unit (50) determines an estimated glucose value greater than the threshold value.

In an aspect according to any one of the preceding aspects the sensor (5), during the activation step of the pump (4) of the power generation procedure, is configured for emitting one or more signals representative of the control parameter of the fluid of the patient (optionally blood and/or interstitial fluid). In an aspect according to the preceding aspect, the sensor (5) is configured for emitting signals representative of the control parameter of the fluid of the patient (optionally blood and/or interstitial fluid) with a time period selectable in the range of 5 minutes to 15 minutes.

In an aspect according to any one of the preceding aspects, the control unit (50) is configured for commanding the pump (4) to be turned off if the estimated value of the control parameter, optionally the estimated value of glucose, is lower than the threshold value of the control parameter.

In an aspect according to any one of the preceding aspects, the control unit (50), during the activation step of the pump (4), is configured for performing at least one of the following sub-steps:
maintaining the pump (4) active for a predetermined period of time,
supplying the predetermined amount of fluid of the patient (optionally blood and/or interstitial fluid) to the biofuel cell.

In an aspect according to any one of the preceding aspects, the power generation procedure also includes a step of determining, based on the comparison between said estimated value of the control parameter and the threshold value, an amount of fluid of the patient (optionally blood and/or interstitial fluid) to be sent to the biofuel cell.

In one aspect according to any one of the preceding aspects, the medical device (1) includes a flow sensor connected to the control unit (50) and configured for emitting a signal representative of the flow rate of fluid of the patient (optionally blood or interstitial fluid) passing through the pump (4).

In an aspect according to any one of the preceding aspects, the power generation procedure also includes the following steps:
receiving the signal from the flow sensor,
based on the signal emitted by the flow sensor, estimating the flow rate of fluid of the patient (optionally blood or interstitial fluid) delivered by the pump (4),
based on the estimated flow rate, commanding the activation of the pump (4) for a predetermined period of time for supplying the estimated amount of fluid of the patient (optionally blood or interstitial fluid) to the biofuel cell (3).

In an aspect according to any one of the preceding aspects, the control unit (50) is also connected to the biofuel cell (3) which is configured for emitting a signal representative of an electrical parameter representative of the electrical energy produced. In an aspect according to any one of the preceding aspects, the power generation procedure performed by the control unit (50), further comprises the following steps:
receiving the signal from the biofuel cell (3),
based on the signal emitted by the biofuel cell (3), estimating a value of the electrical parameter,
comparing the value of the electrical parameter with an electrical threshold value,
based on said comparison, commanding the supply of the electrical energy produced by the biofuel cell (3) to the battery (6).

In an aspect according to any one of the preceding aspects, the control unit (50) is configured for commanding the supply of electrical energy produced by the biofuel cell (3) to the battery (6) when the value of the electrical parameter is greater than the electrical threshold value. In an aspect according to any one of the preceding aspects, the electrical parameter comprises at least one selected in the group of: an electrical voltage measured at the ends of the biofuel cell, an electrical current, an electrical energy, an electrical resistance.

In an aspect according to any one of the preceding aspects, the electrical parameter includes an electrical voltage, wherein the control unit (50), while producing electricity, is configured for commanding the supply of the electrical energy produced by the biofuel cell (3) to the battery (6) when the value of the electrical voltage measured at the ends of the biofuel cell (3) is greater than the electrical threshold value. In one aspect according to the preceding aspect, the electrical threshold value is comprised between 0.2 Volt and 0.4 Volt.

In one aspect according to any one of the preceding aspects the battery (6) is electrically connected to the pump (4) and configured to power it. In an aspect according to any one of the preceding aspects the battery (6) is electrically connected to the sensor (5) and configured to power it. In an aspect according to any one of the preceding aspects the battery (6) is electrically connected to the control unit (50) and configured to power it. In an aspect according to any one of the preceding aspects, the battery (6) is of the solid-state type. In an aspect according to any one of the preceding aspects the battery (6) is of the lithium iodide type.

In an aspect according to any one of the preceding aspects, the medical device (1) comprises an energy converter (7) interposed between the battery (6) and the biofuel cell (3). In an aspect according to any one of the preceding aspects, the energy converter (7) is configured for receiving in input electrical energy produced by the biofuel cell (3) and emitting in output a predetermined electrical energy for charging the battery (6). In one aspect according to the preceding aspect, the electric energy at the output of the energy converter is higher than the input electric energy.

In an aspect according to any one of the preceding aspects, the energy converter (7) includes a voltage regulator configured for receiving in input electrical voltage generated by the biofuel cell (3) and emitting in output a predetermined electrical voltage for charging the battery (6). In one aspect according to the preceding aspect, the electrical voltage at the output of the voltage regulator is comprised between 2 V and 5.5 V. In an aspect according to any of the two preceding aspects the electrical voltage at the output of the voltage regulator is higher than the electrical voltage at the input. In an aspect according to any one of the preceding aspects, the energy converter (7) is configured for emitting in output the predetermined electrical voltage if the input electrical voltage generated by the biofuel cell (3) is higher than a predetermined threshold voltage value. In an aspect according to the preceding aspect said predetermined threshold voltage value is comprised between 0.2 V and 0.4 V.

In an aspect according to any one of the preceding aspects, the control unit (50) is connected to the energy converter (7) and is active in command on the latter for allowing the electrical energy at the output of the energy converter (7) to be sent to the battery (6). In an aspect according to any one of the preceding aspects, the power generation procedure performed by the control unit (50) includes the following steps:
receiving the signal from the biofuel cell (3),
based on the signal emitted by the biofuel cell (3), estimating a value of the electrical parameter,
comparing the value of the electrical parameter with an electrical threshold value,
based on said comparison, commanding the activation of the energy converter (7) for supplying electrical energy produced by the biofuel cell (3) to the battery (6).

In one aspect according to any one of the preceding aspects the pump (4) is of the DC voltage type, optionally it is an electric pump.

In an aspect according to any one of the preceding aspects the medical device (1) comprises at least one first and at least one second needle (11, 12) suitable for allowing the passage of fluid of the patient (optionally blood and/or interstitial fluid). In an aspect according to any one of the preceding aspects, the at least one first needle (11) is in fluid communication, optionally direct fluid communication, with the pump (4) and is configured for allowing withdrawal of the fluid of the patient (optionally blood and/or interstitial fluid) to be sent to the pump (4). In an aspect according to any one of the preceding aspects the at least one first needle comprises at least one micro-needle. In an aspect according to any one of the preceding aspects the micro-needle of said at least one first needle (11) has a length comprised between 100 and 500 μm. In an aspect according to any one of the two preceding aspects the micro-needle of said at least one first needle (11) has a diameter comprised between 5 and 50 μm. In an aspect according to any of the three preceding aspects said at least one first needle (11) has a number of micro-needles greater than 50, optionally comprised between 100 and 5000.

In an aspect according to any one of the preceding aspects the at least second needle (12) is in fluid communication, optionally direct fluid communication, with the biofuel cell (3) and is configured for receiving the fluid of the patient (optionally blood and/or interstitial fluid) arriving from the biofuel cell (3) and feeding it back into the patient. In an aspect according to any one of the preceding aspects the at least one second needle comprises at least one micro-needle.

In an aspect according to any one of the preceding aspects the at least one micro-needle of said at least one second needle (12) has a length comprised between 100 and 500 μm. In an aspect according to any one of the two preceding aspects the micro-needle of said at least one second needle (12) has a diameter comprised between 5 and 50 μm. In an aspect according to any one of the three preceding aspects said at least one second needle (12) has a number of micro-needles greater than 50, optionally comprised between 100 and 5000.

In an aspect according to any one of the preceding aspects the holder has at least one contact surface suitable for contacting the skin of a patient. In an aspect according to any one of the preceding aspects, the holder (2) comprises at least one plaster. In an aspect according to any one of the preceding aspects the holder (2), optionally the plaster, comprises at least one adhesive layer (2b) suitable for directly contacting the skin of a patient. In an aspect according to any one of the preceding aspects the adhesive layer (2b) defines the contact surface of the holder (2). In an aspect according to any one of the preceding aspects the holder (2) has a sheet structure. In an aspect according to any one of the preceding aspects the holder extends in thickness between the contact surface and an opposing support surface. In an aspect according to any one of the preceding aspects the holder (2), optionally the plaster, directly carries said at least one first and second needles, optionally the micro-needles of said at least one first and second needles. In an aspect according to any one of the preceding aspects the plurality of micro-needles of said at least one first needle (11) is distributed on the contact surface of the holder, optionally evenly distributed on said contact surface. In an aspect according to any one of the preceding aspects the plurality of micro-needles of said at least one first needle (11) are arranged on the holder with a density greater than 50 micro-needles per cm², optionally with a density comprised between 50 and 1000 micro-needles per cm². In an aspect according to any one of the preceding aspects the plurality of micro-needles of said at least one second needle (12) is distributed on the contact surface of the holder, optionally evenly distributed on said contact surface. In an aspect according to any one of the preceding aspects the plurality of micro-needles of said at least one second needle (12) are arranged on the holder with a density greater than 50 micro-needles per cm², optionally with a density comprised between 50 and 1000 micro-needles per cm².

In one aspect according to any one of the preceding aspects the control unit (50) includes a microprocessor.

In an aspect according to any one of the preceding aspects the medical device (1) includes a data transmitter (15) connected to the control unit (50) and configured for transmitting data to an external mobile device. In one aspect according to the preceding aspect, the mobile device is a smartphone or tablet.

In one aspect according to any one of the preceding aspects the data transmitter (15) is of the wireless transmission type. In one aspect according to the preceding aspect the data transmitter uses Bluetooth or Wi-Fi technology.

In an aspect according to any one of the preceding aspects the control unit (50) is configured for commanding activation of the data transmitter (15) for transmitting a monitoring signal to the external device representative of the control parameter values. In an aspect according to any one of the preceding aspects, the control unit (50) is active in command on the data transmitter (15). In an aspect according to the preceding aspect the control unit (50) is configured for performing a monitoring procedure comprising the following steps:
receiving one or more signals from the sensor (5),
processing each signal emitted by the sensor (5) and estimating respective values of the control parameter of the fluid of the patient (optionally blood and/or interstitial fluid),
comparing each estimated value of the control parameter with a threshold value, and
based on said comparison, commanding the activation of the data transmitter (15) for transmitting a monitoring signal to the external device representative of the values of the control parameter of the patient's fluid (optionally blood and/or interstitial fluid).

In an aspect according to any one of the preceding aspects the control unit (50) is connected to the battery (6). In an aspect according to the preceding aspect the control unit (50) is configured for performing a safety procedure comprising the following steps:
estimating a charging level of the battery (6),
comparing the charging level of the battery (6) with a threshold value of minimum charge,
in case the charging level of the battery (6) is lower than the threshold value of minimum charge, commanding the activation of the data transmitter (15) for transmitting the value of the charging level of the battery to the external device.

In an aspect according to any one of the preceding aspects, the medical device (1) comprises a charge receiver (16) connected to the battery (6) and configured for receiving electromagnetic waves, optionally microwaves, from an external charger and generating electrical energy. In one aspect according to the preceding aspect the electromagnetic waves are microwaves.

In an aspect according to any of the preceding two aspects, the charge receiver (16), based on the received electromagnetic waves, is configured for transmitting electrical energy to the battery (6) for allowing it to be recharged.

In an aspect according to any one of the preceding aspects, the charge receiver (16) comprises an antenna configured for receiving electromagnetic waves, optionally microwaves, for generating electric current and convert it into a voltage for charging the battery (6).

In an aspect according to any one of the preceding aspects the medical device (1) comprises a charge transmitter (17) connected to the battery (6) and configured for emitting electromagnetic waves suitable for charging an implantable device (101). In an aspect according to any one of the preceding aspects, the charge transmitter (17) comprises a transmitting antenna configured for generating electromagnetic waves, optionally microwaves. In an aspect according to any one of the preceding aspects the charge transmitter (17) is connected to the control unit (50). In an aspect according to any one of the preceding aspects the control unit (50) is active in command on said charge transmitter (17) and configured for:
receiving from the battery (6) a signal representative of the charging level of the battery,
based on the charging level, commanding the activation of the charge transmitter for emitting electromagnetic waves to charge an implantable device (101).

In an aspect according to any one of the preceding aspects the control unit (50) is connected to the battery (6) and the data transmitter (15). In an aspect according to any one of the preceding aspects, the power generation procedure also includes the following steps:
receiving from the battery (6) a signal representative of the charging level of the battery,
process said signal emitted by the battery (6) to estimate a value of the charging level,
compare said value of the charging level with a minimum threshold value of charge,
in case the value of the charging level is lower than the threshold value of minimum charge, commanding the data transmitter (15) for transmitting an alarm signal to an external mobile device to request charging the battery (6).

In one aspect it is provided a charging method of a medical device according to any one of the preceding aspects. In one aspect according to the preceding aspect the method includes the steps of:
detecting, using the sensor (5), a signal representative of a control parameter of the fluid of the patient (optionally blood and/or interstitial fluid),
processing the signal emitted by the sensor (5) and estimating a value of the control parameter of the fluid of the patient (optionally blood and/or interstitial fluid),
comparing said estimated value of the control parameter with a value of a threshold parameter, and
based on said comparison, activating the pump (4) for supplying fluid of the patient (optionally blood and/or interstitial fluid) to the biofuel cell (3) for generating electrical energy.

In an aspect according to the preceding aspect, the pump (4), based on the comparison step, is activated for supplying a predetermined amount of fluid of the patient (optionally blood and/or interstitial fluid) to the biofuel cell. In an aspect according to any one of the three preceding aspects, the control parameter of the fluid of the patient (optionally blood and/or interstitial fluid) includes at least one of: blood glucose, glycated hemoglobin, oxygen concentration, heart rate, blood pressure, temperature, and amino acid or fat concentration. In an aspect according to any one of the preceding method aspects the threshold parameter includes at least one of: blood glucose, glycated hemoglobin, oxygen concentration, heart rate, blood pressure, temperature, amino acid or fat concentration.

In an aspect according to any one of the preceding method aspects the step of estimating the value of the control parameter includes estimating a glucose value in the fluid of the patient (optionally blood and/or interstitial fluid). In an aspect according to any one of the preceding method aspects the step of comparing involves comparing the estimated glucose value with a threshold value. In an aspect according to any one of the preceding method aspects the activation of the pump is performed if the estimated glucose value is higher than the threshold value.

In an aspect according to any one of the preceding method aspects the activation of the pump (4) includes at least one of the following substeps:
activating the pump (4) for a predetermined period of time,
activating the pump (4) for supplying the predetermined amount of fluid of the patient (optionally blood and/or interstitial fluid) to the biofuel cell.

In an aspect according to any one of the preceding method aspects the method includes a step of determining, based on the comparison between said estimated value of the control parameter and the threshold value, a predetermined amount of fluid of the patient (optionally blood and/or interstitial fluid) to be sent to the biofuel cell (3).

In an aspect according to any one of the preceding method aspects the method also includes the following steps:
detecting, using the flow sensor, the flow rate of fluid of the patient (optionally blood and/or interstitial fluid) being delivered by the pump (4),
based on the estimated flow rate, activating the pump (4) for a predetermined period of time for supplying the predetermined amount of fluid of the patient (optionally blood and/or interstitial fluid) to the biofuel cell (3).

In an aspect according to any one of the preceding method aspects the method includes the following steps:
estimating a value of an electrical parameter representative of the electrical energy produced by the biofuel cell,
comparing the value of the electrical parameter with an electrical threshold value,
based on said comparison, commanding the supply of electrical energy produced by the biofuel cell (3) to the battery (6).

In an aspect according to any one of the preceding method aspects supplying electrical energy produced by the biofuel cell (3) to the battery (6) is performed when the value of the electrical parameter is higher than the electrical threshold value. In an aspect according to any one of the preceding method aspects the electrical parameter comprises at least one selected in the group of: a measured electrical voltage at the ends of the biofuel cell, an electrical current, an electrical energy, an electrical resistance. In an aspect according to any one of the preceding method aspects, the electrical parameter comprises an electrical voltage, wherein, during the production of electrical energy, the supply of the electrical energy produced by the biofuel cell (3) to the battery (6) is performed when the value of the measured electrical voltage at the ends of the biofuel cell is higher than the electrical threshold value. In one aspect according to the preceding method aspect the electrical threshold value is comprised between 0.2 Volt and 0.4 Volt.

In an aspect according to any one of the preceding method aspects the method includes the sub-step of supplying, during the production of electricity by the biofuel cell (3), electricity to the converter (7).

In an aspect according to any one of the preceding aspects, the method includes the sub-step of measuring the value of electrical voltage at the ends of the negative and positive poles of the biofuel cell (3). In an aspect according to any one of the preceding aspects, the method includes the sub-step of:
comparing said voltage value with an electrical threshold value,
in case said voltage value is higher than the electrical threshold value, activating the converter (7) for supplying electricity to the battery (6).

In an aspect according to any one of the preceding aspects, the method comprises a step of transmitting data to an external device, via the transmitter. In an aspect according to any one of the preceding aspects, the transmitter comprises a transmitting antenna suitable for communicating via Bluetooth or Wi-Fi technology.

In an aspect according to any one of the preceding aspects, the steps of the power generation procedure are performed by a control unit (50).

In one aspect, it is provided a method for treating a fluid of a patient (optionally blood and/or interstitial fluid) using a medical device (1) including:
a holder (2) applicable on the skin of a patient (P),
at least one rechargeable battery (6) carried by the holder (2),
at least one biofuel cell (3) carried by the holder (2) and connected to the battery (6), wherein the biofuel cell (3) is configured for receiving fluid of the patient (optionally blood and/or interstitial fluid) from the patient (P) and producing electrical energy that may be used to charge the battery (6),
at least one pump (4) carried by the holder (2) and configured for supplying fluid of the patient (optionally blood and/or interstitial fluid) to the biofuel cell (3),
at least one sensor (5) carried by the holder (2) and configured for emitting a signal representative of a control parameter of a fluid of the patient (optionally blood and/or interstitial fluid),
a control unit (50) connected to the pump (4) and the sensor (5) and active in control on said pump (4), wherein said method of treating fluid of the patient (optionally blood and/or interstitial fluid) includes the steps of:
detecting, using the sensor (5), a signal representative of a control parameter of the fluid of the patient (optionally blood and/or interstitial fluid),
processing the signal emitted by the sensor (5) and estimating a value of the control parameter of the fluid of the patient (optionally blood and/or interstitial fluid),
comparing said estimated value of the control parameter with a threshold value, and
based on said comparison, activating the pump (4) for supplying fluid of the patient (optionally blood and/or interstitial fluid) to the biofuel cell (3).

In one aspect according to the preceding aspect, the biofuel cell (3), following the activation of the pump, receives a flow of fluid of the patient (optionally blood and/or interstitial fluid) to treat it and producing electricity.

In one aspect according to any one of the preceding aspects the medical device (1) used for performing the method of treating the fluid of the patient (optionally blood and/or interstitial fluid) is according to any one of the preceding aspects.

In an aspect according to any one of the preceding aspects, the pump (4), based on the comparison step, is activated for supplying a predetermined amount of fluid of the patient (optionally blood and/or interstitial fluid) to the biofuel cell.

In an aspect according to any one of the preceding aspects, the control parameter of the fluid of the patient (optionally blood and/or interstitial fluid) includes at least one of: blood glucose, glycated hemoglobin, oxygen concentration, heart rate, blood pressure, temperature, amino acid or fat concentration.

In an aspect according to any one of the preceding method aspects the step of estimating the value of the control parameter includes estimating a glucose value in the fluid of the patient (optionally blood and/or interstitial fluid). In an aspect according to any one of the preceding method aspects the step of comparing involves comparing the estimated glucose value with a threshold value. In an aspect according to any one of the preceding method aspects the activation of the pump is performed if the estimated glucose value is higher than the threshold value. In an aspect according to any one of the preceding aspects, the biofuel cell, following the activation of the (4), is configured for generating electrical energy using glucose present in the flow of fluid of the patient (optionally blood and/or interstitial fluid) passing through the biofuel cell. In an aspect according to any one of the preceding aspects, the biofuel cell (3) reduces the amount of glucose in the flow of fluid of the patient (optionally blood and/or interstitial fluid) entering the biofuel cell. In an aspect according to any one of the preceding aspects, the biofuel cell uses glucose in the flow of fluid of the patient (optionally blood and/or interstitial fluid) flowing through the cell for generating energy for feeding back to the patient a flow of fluid of the patient (optionally blood and/or interstitial fluid) having a glucose percentage lower than the glucose percentage in the flow of fluid of the patient (optionally blood and/or interstitial fluid) entering the biofuel cell.

In an aspect according to any one of the preceding method aspects the activation of the pump (4), it includes at least one of the following sub-steps:
activating the pump (4) for a predetermined period of time,
activating the pump (4) for supplying the predetermined amount of fluid of the patient (optionally blood and/or interstitial fluid) to the biofuel cell.

In an aspect according to any one of the preceding aspects, the method includes a step of determining, depending on the comparison between said estimated value of the control parameter and the threshold value, a predetermined amount of fluid of the patient (optionally blood and/or interstitial fluid) to be sent to the biofuel cell (3). In an aspect according to any one of the preceding aspects, the method also includes the following steps:
detecting, using the flow sensor, the flow rate of fluid of the patient (optionally blood and/or interstitial fluid) being delivered by the pump (4),
based on the estimated flow rate, activating the pump (4) for a predetermined period of time for supplying the predetermined amount of fluid of the patient (optionally blood and/or interstitial fluid) to the biofuel cell (3).

In one aspect, it is provided an implantable device (101) applicable under the skin of a patient (P) for charging an implantable electronic component (110), said implantable device (101) comprising:
at least one battery (102),
a charge receiver (103) connected to the battery (102), said charge receiver (103) being configured for supplying electrical energy to the battery (102) to be charged.

In an aspect according to the preceding aspect, the implantable device (101) includes at least one charge transmitter (107) connected to the battery (102) and configured for transmitting electrical energy to the implantable electronic component (110) to recharge it.

In one aspect according to any one of the preceding two aspects the charge receiver (103) of the implantable device is configured for receiving electromagnetic waves and converting them into electrical energy to be sent to the battery (102) of the implantable device. In an aspect according to any one of the preceding aspects, the charge receiver (103) of the implantable device is configured for receiving electromagnetic waves, optionally microwaves, from an external charging source (104) and generating electrical energy. In an aspect according to any one of the preceding aspects, the charge receiver (103) of the implantable device includes an antenna configured for receiving electromagnetic waves for generating electrical energy and converting it into voltage for charging the battery (102) of the implantable device.

In an aspect according to any one of the preceding aspects, the implantable device (101) comprises a control unit (500) connected to the battery (102) and activated in command on the charge transmitter (107). In an aspect according to any one of the preceding aspects, the battery (102) is configured for emitting a signal representative of a charging level of the battery (102).

In one aspect according to any one of the two preceding aspects, the control unit (500) is configured for performing a charging procedure of the implantable electronic component including the step of receiving one or more signals from the battery (102) representative of the charging level.

In one aspect according to the preceding aspect, the charging procedure of the implantable electronic component includes the step of processing each signal emitted by the battery (102) and estimating respective values of the charging level of the battery (102).

In an aspect according to the preceding aspect the charging procedure of the implantable electronic component includes a step of comparing each estimated value of the charging level of the battery (102) with a threshold value of minimum charging. In an aspect according to the preceding aspect, the threshold value of minimum charging is comprised between 20% and 30% of a total accumulable energy of the battery (102).

In an aspect according to any one of the two preceding aspects, the activation of the charge transmitter (107) for supplying electrical energy stored in the battery (102) to the implantable electronic component (110) is performed in case the estimated value of the charging level is higher than the threshold value of minimum charge.

In an aspect according to any one of the preceding aspects, the charge transmitter (107) comprises at least one voltage regulator configured for receiving as input at least part of the electrical energy stored in the battery (102). In an aspect according to any one of the preceding aspects, the voltage regulator is configured for supplying a predetermined amount of electrical energy to the implantable electronic component (110). In an aspect according to any one of the preceding aspects, the voltage regulator is configured for emitting a predetermined electrical output voltage comprised between 2.5 Volt and 5 Volt for charging the implantable electronic component (110).

In an aspect according to any one of the preceding aspects, the implantable device includes a data communication module (150) connected to the control unit (500) and configured for transmitting data to an external mobile device (120) connectable to the implantable device (101). In one aspect according to the preceding aspect, the external mobile device (120) is a smartphone or tablet. In an aspect according to any one of the preceding aspects, the data communication module (150) is wireless. In one aspect according to the preceding aspect the data communication module (150) uses a Bluetooth or Wi-Fi technology. In an aspect according to any one of the preceding aspects, the control unit (500) is configured for commanding the data communication module (150) to send an alarm signal to the external mobile device (120) if the battery value of the charging level (102) is below the threshold value of minimum charge. In an aspect according to any one of the preceding aspects, the control unit (500) comprises a microprocessor.

In one aspect is provided a medical system (100) comprising:
at least one implantable device (101) applicable under the skin of a patient (P), wherein said implantable device includes a battery (102) configured for supplying the implantable device itself,
at least one charging source (104) configured for providing electrical energy to the battery (102) of the implantable device.

In an aspect according to the preceding aspect, the implantable device (101) includes a charge receiver (103) connected to the battery (102) of the implantable device (101) and configured for supplying electrical energy to the battery (102) for recharging it. In an aspect according to the preceding aspect, the charging source is configured for providing electrical energy to the charge receiver (103) of the implantable device (101) for recharging the battery (102) of the implantable device.

In one aspect according to any one of the preceding aspects the charging source (104) comprises a transmitting antenna configured for generating electromagnetic waves. In one aspect according to any one of the preceding aspects the medical system includes an implantable electronic component (110) configured for:
monitoring the activity of at least one organ of the patient, and/or
working on an organ of the patient.

In an aspect according to any one of the preceding aspects the implantable electronic component (110) includes at least one of: a pacemaker, a cardiac defibrillator, a sacral nerve stimulator, a glucose meter, gastric stimulator. In an aspect according to any one of the preceding aspects, the implantable electronic component (110) comprises an electrical energy storage device (111) configured for supplying electrical components of the implantable electronic component (110) itself. In an aspect according to any one of the preceding aspects, the implantable device (101) is connected to the implantable electronic component (110). In an aspect according to any one of the preceding aspects, the implantable device (101) is directly connected to the implantable electronic component (110) via an electrical cable. In an aspect according to any one of the preceding aspects the implantable device is according to any one of the preceding aspects. In an aspect according to any one of the preceding aspects the control unit (500) of the implantable device (101) is connected to the electric energy accumulator (111) of the implantable electronic component (110). In an aspect according to any one of the preceding aspects the control unit (500) of the implantable device (101) is configured for estimating one or more values of the charging level of the electric energy accumulator (111) of the implantable electronic component (110). In an aspect according to any one of the preceding aspects, the control unit (500) of the implantable device (101) is configured for:
comparing each estimated value of the charging level of the electric energy accumulator (111) with a respective threshold value of minimum charging,
based on the charging level of the electric energy accumulator (111), commanding the supply of electric energy stored in the battery (102) of the implantable device (101) to the electric energy accumulator (111).

In an aspect according to the preceding aspect the threshold value of minimum charge is comprised between 20% and 30% of a total accumulable energy by the electric energy accumulator (111). In an aspect according to any one of the preceding aspects, the control unit (500) of the implantable device (101), in the case the charging level of the electric energy accumulator (111) is lower than the threshold value of minimum charge and in the case the estimated value of the charging level of the battery (102) of the implantable device (101) is greater than the threshold value of minimum charge is configured for commanding the activation of the charge transmitter (107) for supplying electrical energy stored by the battery (102) of the implantable device (101) to the electric energy accumulator (111) of the implantable electronic component (110).

In one aspect according to any one of the preceding aspects, the charging source (104) includes a wireless charging base external to the patient or a wearable medical device (1) wearable on the skin of the patient.

In an aspect according to any one of the preceding aspects the medical device (1) comprises a charge transmitter (17) connected to the battery (6) of the medical device (1) and configured for emitting electromagnetic waves usable for charging the battery (102) of the implantable device (101). In an aspect according to any one of the preceding aspects, the charging source (104) comprises at least one medical device (1) according to any one of the preceding aspects. In an aspect according to any one of the preceding aspects the medical device (1) is connectable to the implantable device (101) for charging of the battery (102) of the latter.

In an aspect according to any of the preceding two aspects the charge receiver (103) of the implantable device (101) is configured for receiving electromagnetic waves transmitted by the charge transmitter (17) of the medical device (1). In an aspect according to the preceding aspect, the charge receiver (103) of the implantable device is configured for converting the electromagnetic waves received by the charge transmitter (17) of the medical device (1) into electrical energy to be supplied to the battery (102) of the implantable device (101).

In an aspect according to any one of the preceding aspects the charge receiver (103) of the implantable device (101) includes an antenna configured for receiving electromagnetic waves for generating electrical energy. In an aspect according to the preceding aspect, the antenna is configured for converting the received electromagnetic waves into voltage for charging the battery (102) of the implantable device.

In one aspect according to any one of the preceding aspects of medical system the implantable device (101) includes at least one of: a pacemaker, cardiac defibrillator, sacral nerve stimulator, glucose meter, glucose delivery pump, infusion pump for medical treatments, cardiac mechanical assist device.

In an aspect according to any one of the preceding aspects, the implantable device (101) comprises a control unit (500) connected to the battery (102) of the implantable device and configured for receiving from the latter a signal representative of its charging level. In an aspect according to any one of the preceding aspects the charging source (104) is connected to and electrically supplies the control unit (500).

In one aspect there is provided a method of recharging a medical system according to any one of the preceding aspects of medical system. In an aspect according to the preceding aspect the recharging method includes the steps of:
estimating one or more values of the charging level of the electric energy accumulator (111),
comparing the estimated value of the charging level of the electric energy accumulator (111) with a respective threshold value of minimum charge,
at least based on said comparison, commanding the supply of electrical energy stored in the battery (102) of the implantable device (101) to the electric energy accumulator (111) of the implantable electronic component (110). In an aspect according to the preceding aspect the threshold value of minimum charge is comprised between 20% and 30% of a total energy accumulable by the electric energy accumulator (111). In an aspect according to any one of the three preceding aspects, the step of commanding the supply of the electrical energy accumulated by the battery (102) of the implantable device (101) to the electric energy accumulator (111) of the implantable electronic component (110) involves the sub-steps of:
estimating one or more values representative of the charging level of the battery (102) of the implantable device (101),
comparing each estimated value of the charging level of the battery (102) of the implantable device (101) with a threshold value of minimum charge.

In an aspect according to the preceding aspect, the threshold value of minimum charge is comprised between 20% and 30% of a total energy accumulable by the electric energy accumulator (111). In an aspect according to any one of the preceding aspects the step of commanding the supply of the electric energy accumulated by the battery (102) to the implantable electronic component (110) is performed if:
each value of the charging level of the battery (102) of the implantable device (101) is higher than the threshold value of minimum charge,
each value of the charging level of the accumulator (111) of the implantable electronic component (110) is lower than the respective threshold value of minimum charge.

In an aspect according to any one of the preceding aspects, the method comprises a step of sending an alarm signal to an external mobile device (120) if each value of the charging level of the battery (102) of the implantable device (101) is lower than a respective threshold value of minimum charge. In an aspect according to any one of the preceding aspects, the method includes, following the step of sending the alarm signal to the mobile device (120), a step of placing the charging source (104) at a distance from the charge receiver (103) of the implantable device (101) comprised between 5 mm and 30 mm, for transferring energy from the charging source (104) to the implantable device (101). In an aspect according to any one of the preceding aspects, the method includes a step of sending an alarm signal to the medical device (1) if each value of the charging level of the battery (102) is lower than the threshold value of minimum charge. In an aspect according to any one of the preceding aspects, the method, following the step of sending the alarm signal to the medical device (1), comprises the steps of:
receiving one or more signals representative of the charging level of the battery (2) of the medical device,
processing each signal emitted from the battery (2) of the medical device (1) and estimating respective values of the charging level,
comparing each estimated value of the charging level of the battery (2) of the medical device (1) with a threshold value of minimum charge of the medical device,
based on said comparison, commanding the transfer of electrical energy from the medical device (1) to the implantable device (101).

In an aspect according to the preceding aspect the threshold value of minimum charging level of the medical device is comprised between 20% and 30% of a total energy accumulable by said battery (2). In an aspect according to any one of the preceding aspects, the step of commanding the transfer of electrical energy from the medical device (1) to the implantable device (101) is performed if the estimated value of the charging level of the battery (2) of the medical device (1) is higher than the respective threshold value of minimum charge of the medical device. In an aspect according to the preceding aspect, the energy transfer from the medical device (1) toward the implantable device (101) is performed by means of the charge transmitter (17) of the medical device.

### BRIEF DESCRIPTION OF THE DRAWINGS

Some embodiments and some aspects of the invention will be described below with reference to the accompanying drawings, provided for illustrative purposes only and therefore not limiting wherein:

FIG. 1 is a schematic view of a medical device applied on a patient's skin;

FIG. 2 is a schematic view of the medical device of FIG. 1;

FIG. 3 is a schematic view of a medical system including a medical device according to the present invention;

FIG. 4 is a schematic view of a variant of a medical system including a medical device according to the present invention;

FIG. 5 is a flow chart showing the steps implementable by a medical device according to the present invention;

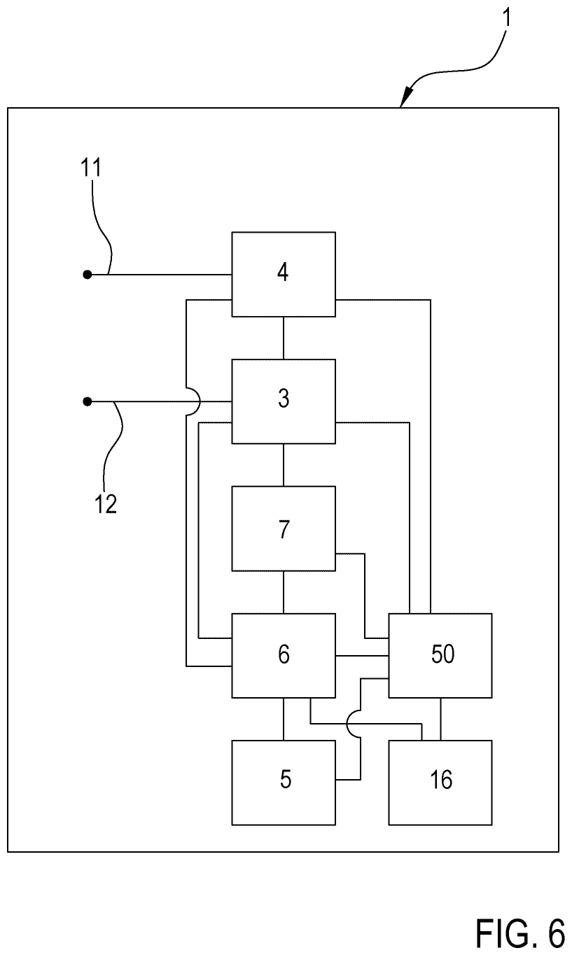

FIG. 6 is a further schematic view of a medical device according to the present invention;

FIGS. 7 and 8 are respective schematic views of variations of the medical system of FIG. 4;

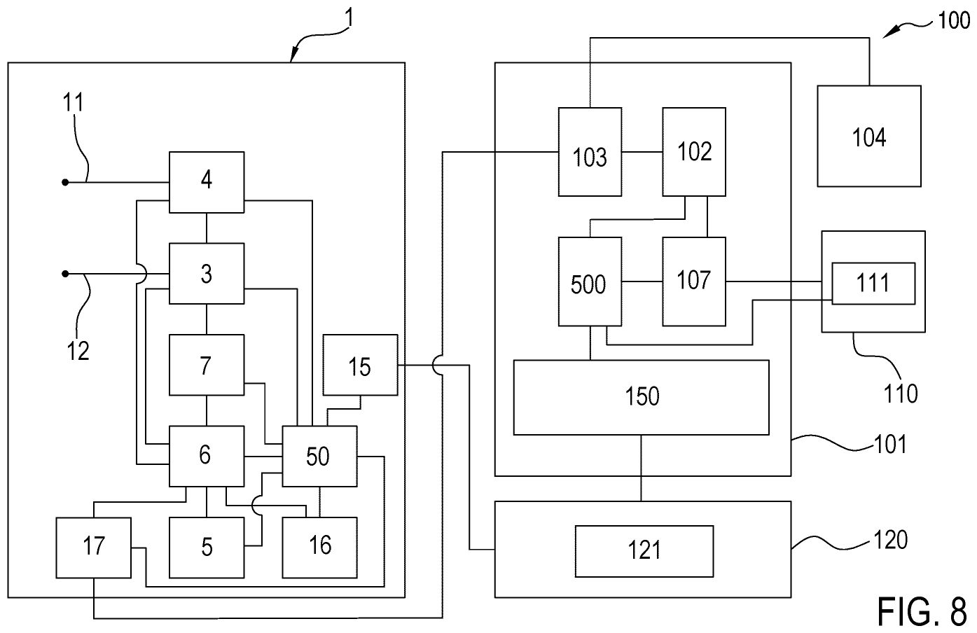

FIG. 9 is a flow chart showing the steps implementable by a medical system.

### DEFINITIONS AND CONVENTIONS

Note that in the present detailed description the parts illustrated in the various figures are shown with the same numerical references. The figures may illustrate the subject matter of the invention by means of representations that are not to scale; therefore, parts and components illustrated in the figures related to the subject matter of the invention may relate only to schematic representations.

At least one of the medical device, implantable device, and medical system described below may include/use at least one control unit responsible for controlling operating conditions performed by the same medical device, implantable device, or the medical system and/or for controlling method steps executable by them. The control unit may be a single unit or consist of a plurality of distinct control units depending on design choices and operational requirements.

By control unit is meant an electronic component which may include at least one of: a digital processor (CPU), an analog type circuit, or a combination of one or more digital processors with one or more analog type circuits. The control unit may be “configured” or “programmed” to perform certain steps: this may be accomplished in practice by any means that allows the control unit to be configured or programmed. For example, in the case of a control unit comprising one or more CPUs and one or more memories, one or more programs may be stored in appropriate memory banks attached to the CPU(s); the program(s) contain instructions that, when executed by the CPU(s), program or configure the control unit to perform the operations described in relation to the control unit. Alternatively, if the control unit is/includes analog type circuitry, then the circuitry of the control unit may be designed to include circuitry configured, in use, to process electrical signals such that to perform the steps related to the control unit. Portions of the process described herein may be accomplished by means of a data processing unit, or control unit, that is technically replaceable with one or more electronic processors designed to execute a portion of software program or firmware loaded into a memory medium. Such software program may be written in any programming language of known type. The electronic processors, if two or more in number, may be interconnected by means of a data connection such that their computational powers are shared; the same electronic processors may thus be installed in geographically different locations, realizing through the aforementioned data connection a distributed computing environment. The data processing unit, or control unit, may be a general purpose processor configured to perform one or more parts of the process identified in the present disclosure through the software program or firmware, or may be an ASIC or dedicated processor or FPGA, specifically programmed to perform at least part of the operations of the process described herein.

The memory medium may be non-transitory and may be internal or external to the processor, or control unit, or data processing unit, and specifically may be a memory geographically located remote from the processor. The memory medium may also be physically divided into multiple portions, or in cloud form, and the software program or firmware may be stored on geographically divided portions of memory.

The biofuel cell is configured to produce electricity through the chemical transformation of a biological substrate. The biofuel cell used in the medical device 1 and/or medical system 100 may be of enzymatic type i.e., using enzymes as catalysts for allowing the occurrence of chemical reactions for producing electricity. The enzymatic biofuel cell may perform a chemical transformation to obtain electricity under physiological reaction conditions (in the range of 37° C. and basically under pH-7 conditions). The enzymes usable in the biofuel cell are: glucose oxidase (GOx), glucose dehydrogenase (GDH), bilirubin oxidase (BOD), and laccase. The biofuel cell may take advantage of direct electron transfer (DET) through the use of nanostructures, in particular, carbon nanostructures (CNTs). The electrodes (anode and cathode) of the biofuel cell may be arranged in porous bodies, communicating with the blood through a selective membrane (e.g., a glucose selective membrane for the anode and an oxygen selective membrane for the cathode). The electrodes may also be separated from each other, on the inner side, that is, on the side where the electrodes face each other, by a selective membrane for ions (H+) that, by osmosis, may pass from the anode to the cathode for the formation of water.

The term ‘implantable’ refers to a device and/or system that may be at least partially implanted under a patient's skin.

### DETAILED DESCRIPTION

### Medical Device

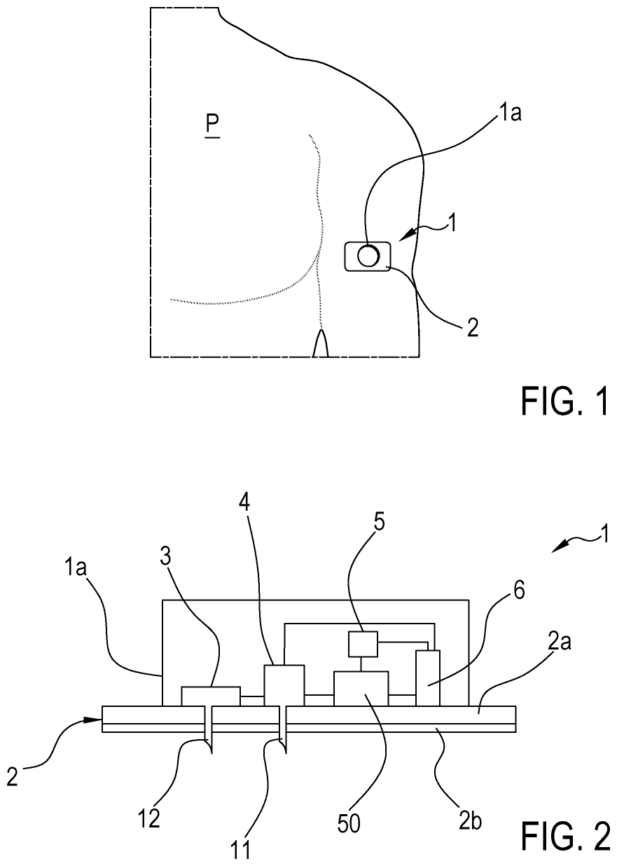

A medical device applicable to the skin of a patient P has been collectively referred to as 1. The device uses one or more substances in the fluid of the patient (optionally biological fluid) to produce electricity. The medical device 1 is designed for external application and, as shown in FIG. 1, may include a holder 2, such as a plaster, applicable on the skin of patient P over a large, linear area, such as the torso, back, arms, and thighs. To produce electricity, the medical device 1 monitors and exploits one or more substances, such as glucose, through a noninvasive technique that allows data acquisition without the need to interact directly with the biological environment of the patient.

The holder 2 may have a multilayer structure, for example defined by at least one or more upper or rigid layers 2a, overlapped to a lower layer or adhesive layer 2b suitable for contacting the skin of the patient. The adhesive layer 2b may be made of elastic and flexible material to fit as closely as possible to the skin surface where it is applied to maximize adherence. Each rigid layer is carried by the adhesive layer on the side opposite the skin, for supporting one or more of the subsequently detailed electrical or electronic components of the medical device 1.

The holder 2 may have a rectangular conformation (FIG. 1) of a suitable size for allowing its application on the arms; for example, the holder 2 may have a contact surface suitable for being placed in contact with the skin of a patient having a surface area comprised between 20 cm² and 150 cm². In fact, the contact surface is essentially defined by the adhesive layer 2b. However, it is not excluded use of a holder 2 having a different shape (e.g., circular or square) or different dimensions, varying according to the application points of the medical device 1 itself. From a material point of view, the holder 2 may be made of sterile, skin-friendly and hypoallergenic material, e.g., cotton (in the case of adhesive layer 2b) or rigid synthetic polymers (in the case of rigid layer 2a).

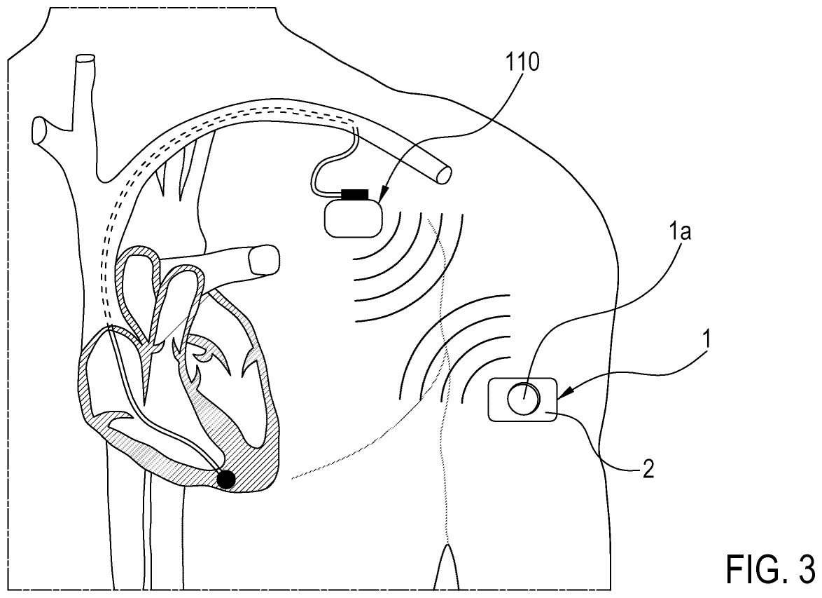

The medical device 1 may include a case 1a stably engaged to the holder 2 for housing one or more electrical/electronic components of the medical device 1 itself which will be better detailed later. In the example shown in FIG. 2, the case 1a may has a cylindrical or hemispherical conformation. In particular, the case 1a may define, in cooperation with the holder 2, a watertight internal volume, completely separated from an external environment and capable of preventing the passage of gases or fluids.

The medical device 1 includes at least one sensor 5 carried by the holder 2, e.g. housed in the inner volume of the case 1a, configured for monitoring a control parameter representative of a fluid of the patient P, optionally biological fluid). In the following description, the term fluid of the patient or biological fluid of the patient is referred to blood or interstitial fluid. The sensor 5 may be configured for emitting a signal representative of the control parameter, for example related to the percentage of glucose in the fluid of the patient or a percentage of glycated hemoglobin, and sending that signal to a control unit 50 detailed below.

The sensor 5 may implement one or more techniques for glucose measurement, such as: occlusion/diffusion spectroscopy, optical coherence tomography, bioimpedance spectroscopy, millimeter wave detection, microwave detection, and high-frequency wave detection. Depending on the glucose measurement technique adopted, the sensor 5 may include: an infrared sensor, a luminescence sensor, an electromagnetic radiation sensor, an acoustic sensor, an ultrasonic sensor, an impedance measurement circuit, and a radio frequency sensor.

In an embodiment, the sensor 5 is placed in contact with the skin of the patient and implements, in a non-limiting manner, a bioimpedance spectroscopy technique, which involves measuring the impedance from the patient's body to the passage of an alternating electric current at low intensity (e.g., equal to 800 μA) and at a predetermined frequency.

For the generation of the signal of the control parameter representative of the glucose concentration in the fluid of the patient, the aforementioned technique, uses the permittivity and conductivity properties of red blood cells; in particular, the implemented technique allows for the determination of a change in the glucose level following the detection of a change in red blood cells in the blood. The sensor 5 may have one or more electrodes placed in contact with or near the skin of the patient P configured for emitting, as mentioned above, an electric current signal to estimate the percentage of glucose present in a composition of the fluid of the patient.

The medical device 1 also includes a battery 6 carried by the holder 2, e.g. also housed in the inner volume of case 1a; the battery 6 is electrically connected to the sensor 5 and configured to power it. The battery 6 may be a rechargeable battery of the solid-state type, optionally lithium iodide, or a conventional lithium-ion battery. The battery 6 has a charging capacity comprised between 0.5 Ampere-hour and 10 Ampere-hour and is configured for supplying a continuous output voltage, for example, comprised between 2.5 Volt and 5 Volt, optionally substantially equal to 3.3 Volt.

The battery 6 is connected to the control unit 50 for its power supply; in fact, the battery 6 is used as the power supply component of the device that may supply power to both the sensor 5 and the control unit 50. The control unit 50, connected to the battery 6, is configured for estimating a value of a charging level of the battery itself. In particular, the control unit 50 is configured for performing a safety procedure comprising the steps of:
estimating a charging level of the battery 6,
comparing the estimated charging level of the battery 6 with a threshold value of minimum charge, for example comprised between 20% and 30% of the total electrical energy that may be stored in the battery 6,
if the value of the charging level of the battery 6, estimated by the control unit 50, is lower than the threshold value of minimum charge, the same control unit 50 may be configured for commanding a data transmitter 15 (later detailed), for emitting an alarm signal to warn the patient of a low charging level of the battery 6.

As will be better described later, the medical device 1 may include at least one biofuel cell 3 configured for using substances in the fluid of the patient to produce electricity; this energy may be used by the device to charge the battery 6.

Thus, the medical device 1 may include an energy converter 7 carried by the holder 2, in particular the rigid layer 2a, and interposed between the biofuel cell 3 and the battery 6. In detail, the energy converter is housed in the inner volume of the case 1a. The energy converter 7 is configured for receiving electrical energy emitted from the biofuel cell 3, convert it, and supplying electrical energy to the battery 6 at a predetermined voltage for charging the battery 6 itself. For example, the energy converter 7 may include a voltage regulator connected to the battery 6 and configured to supply in output a constant electrical voltage greater than an input electrical voltage, which is also constant. The voltage regulator may be a conventional DC-DC power converter, such as a step-up regulator, a buck-boost converter, or a Sepic converter, having an input voltage comprised between 0.5 Volt and 5.5 Volt, and a constant output voltage of essentially 3.3 Volt. The energy converter 7, optionally the voltage regulator, is configurable between:
an active condition wherein it supplies a constant output voltage as indicated above, and
a deactivated condition wherein it does not supply output voltage.

The control unit 50 may be electrically connected to the energy converter 7 and configured for:
determining an input voltage value to the energy converter 7 itself (equivalent to a voltage value measured at the ends of the biofuel cell 3),
comparing the input voltage to energy converter 7 with an electrical threshold value, optionally comprised between 0.2 Volt and 0.4 Volt,
   in case the input voltage is higher than the electrical threshold value, the control unit 50 is configured for commanding the active condition of the energy converter 7 and allowing electricity to be supplies to the battery 6,
   in case the input voltage is lower than the electrical threshold value, the control unit 50 is configured for commanding the deactivated condition of the energy converter 7.

In one variant, the transition between the active and deactivated condition of the energy converter 7 may be internally managed by the converter itself depending on a value of the input voltage; in such a variant, it is not the control unit 50 that controls the transition between the active and deactivated condition but this is managed directly by the energy converter through dedicated circuitry implementing digital or analog logic. If the input voltage to the energy converter 7 is lower than the electrical threshold value, the voltage regulator remains in the deactivated condition, while the energy converter 7 switches its state from the deactivated condition to the active condition if the input voltage is within or exceeds the electrical threshold value (optionally defined between 0.2 Volt and 0.4 Volt).

The energy converter 7 may also include a temperature sensor configured for generating a signal representative of the temperature of the external environment surrounding the energy converter 7 (optionally the voltage regulator). The energy converter 7 may have a circuit component suitable for receiving the temperature signal sent by the temperature sensor and estimating a respective temperature value. The circuit component, depending on the estimated temperature value, is configured for controlling the transition between the active and deactivated condition of the energy converter 7 if the estimated temperature value exceeds a predetermined temperature range, optionally comprised between 39° C. and 50° C. This feature of the energy converter 7 ensures its operation in prefixed temperature range, thus limiting overload or overtemperature conditions that may cause malfunctions or failures of the medical device 1. It should also be noted how the above temperature values may be variable in the specified range depending on the extent of the skin surface of the patient.

As mentioned above, the medical device 1 includes a biofuel cell 3 carried by the holder 2, e.g., also arranged in the inner volume of the case 1a; the biofuel cell 3 is configured for producing electrical energy, e.g., intended for charging the battery 6. The biofuel cell 3 is electrically connected to the energy converter 7 for charging the battery 6 and configured to generate an output voltage signal directed to the energy converter 7: the input voltage to energy converter 7 coincides with the output voltage from biofuel cell 3.

The biofuel cell 3 is configured for producing electrical energy through the chemical transformation of the biological fluid of the patient (as mentioned above through the treatment of the blood and/or interstitial fluid of the patient). In an example, the biofuel cell 3 may be an enzymatic cell suitable for performing chemical transformation to obtain electrical energy, such as using enzymes as a catalyst to oxidize its fuel. The enzymatic biofuel cell is suitable for using at least one of the following enzymes to produce electricity: glucose oxidase (GOx), bilirubin oxidase (BOD), laccase, pyrroloquinoline quinone (PQQ), and glucose dehydrogenase (GDH), which ensure the oxidation of various monosaccharides and disaccharides, e.g., glucose, maltose, lactose, galactose, xylose, and mannose.

The biofuel cell 3 has a case wherein one or more chambers are defined for the passage of a biological fluid of the patient (e.g., blood), each of which is placed in fluid communication with one or more successively detailed needles or micro-needles, which are in charge of withdrawing and re-injecting the blood into the patient. The biofuel cell 3 also has a first and second electrode (anode and cathode), housed in a chamber of the case in contact with the blood and configured for reacting with the latter for producing energy. The first and second electrodes have a platelike conformation (e.g., square or rectangular), having respective inner surfaces mutually facing each other and respective outer surfaces, opposite the inner surfaces, configured for contacting the blood. The first electrode has a selective membrane, e.g., glucose selective, carried by the outer surface and configured for contacting the biological fluid, while the second electrode has an oxygen selective membrane carried by the respective outer surface of the second electrode, also configured for contacting the biological fluid. The biofuel cell 3 also includes a hydrogen ion selective membrane (H⁺) defined in interposition between the respective inner surfaces of the first and second electrodes.

From the materials point of view, each electrode may have either a carbon structure or a carbon nanotube (CNT) structure, such as made of Buckypaper and coated with one of the following enzymes: glucose oxidase (GOx), bilirubin oxidase (BOD), laccase, pyrroloquinoline quinone (PQQ) and glucose dehydrogenase (GDH). The respective carbon nanostructures of the first and second electrodes may be respectively coated with pyrroloquinoline quinone-glucose dehydrogenase (PQQ-GDH) and laccase.

The aforementioned enzymes allow the first and second electrodes to produce electrical energy through the oxidation of a substance in the biological fluid, for example, the oxidation of glucose to gluconolactone, and the reduction of oxygen to water. These reactions generate a potential difference at the ends of the first and second electrodes (open circuit potential), which is proportional to the voltage signal output from the biofuel cell 3.

As mentioned, the medical device 1 may also comprise a first and second needle 11, 12, carried by the holder 2, optionally by the rigid layer 2a, and crossing the adhesive layer 2b (FIG. 2) to fit under the skin of the patient. For example, the first and second needles 11, 12 may be in the form of micro-needles having respective channels for the passage of biological fluid, each having a diameter comprised between 5 μm and 50 μm. The first and second needles 11, 12 are configured for allowing biological fluid to be withdrawn from the patient, allow it to pass through the biofuel cell 3, and feed the fluid back into the patient. In terms of materials, the first and second needles 11, 12 may both be made of a glassy carbon structure or single-crystalline silicon.

In detail, the first needle 11 comprises a plurality of micro-needles each of which has a length comprised between 100 and 500 μm, optionally a diameter comprised between 5 and 50 μm; in greater detail, said at least one first needle 11 has a number of micro-needles greater than 50, optionally between 100 and 5000, distributed on the holder 2, optionally uniformly, with a density greater than 50 micro-needles per cm². Similarly, the second needle 12 comprises a plurality of micro-needles each of which has a length comprised between 100 and 500 μm, optionally a diameter comprised between 5 and 50 μm; in greater detail, said at least one second needle 12 has a number of micro-needles greater than 50, optionally comprised between 100 and 5000, distributed on holder 2, optionally uniformly, with a density greater than 50 micro-needles per cm².

The plurality of micro-needles of the first and second needles are carried by the holder and emerging from the adhesive layer; in detail, the plurality of micro-needles crosses the thickness of the holder such that they carry the fluid of the patient to the components of the medical device designed to receive said fluid.

The use of a plurality of micro-needles rather than a single needle in inlet and a single needle in outlet, may allow the withdrawal of interstitial fluid from different areas of adipose tissue, increasing the accuracy of blood glucose measurements and reducing the variability of results. Furthermore, since the plurality of micro-needles defines a larger fluid withdrawal area than the area covered by a single needle; thus, the flow of interstitial fluid is more evenly distributed than with a single needle, preventing the formation of pockets of stagnant interstitial fluid that could compromise the accuracy of measurements. Additionally, the presence of a plurality of micro-needles allows interstitial fluid to be withdrawn continuously and with a constant flow, as opposed to a single needle in inlet and only one needle in outlet, which may cause unstable and variable flow. This ensures a constant supply of glucose to the biofuel cell, enabling constant power generation. Although the presence of micro-needles allows for several advantages, the use of at least one needle (11, 12) also allows the capabilities of the medical device described above to be exploited for the production of electricity and thus the charging of the device itself.

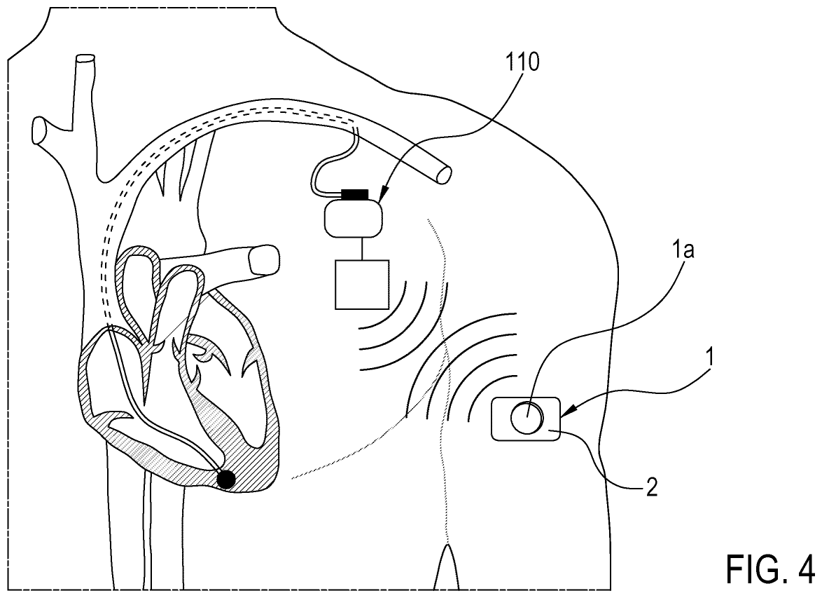

The medical device 1 includes a pump 4 carried by the holder 2, in particular by the top layer 2a of the holder, and housed in the inner volume of the case 1a. As, for example, shown in FIG. 3, the pump 4 is in fluid communication with said first and second needles 11, 12 and with the biofuel cell 3; the pump 4 is configured to allow fluid, e.g., blood, to be withdrawn from the patient's biological environment via the first needle 11, sent to biofuel cell 3 and then returned to the patient's biological environment via the second needle 12. In fact, each needle is placed in fluid communication with the pump 4, through which the biological fluid is passed through the biofuel cell 3: the first needle 11 is configured for withdrawing biological fluid and supplying it to the pump 4, while the second needle 12 is in fluid communication with the biofuel cell 3 and is configured for receiving the biological fluid passing through the cell and feeding it back into the patient.

The pump 4 is electrically powered by the battery 6 and configurable at least between:
an activation condition wherein the pump 4 moves the biological fluid of the patient (e.g., blood) from the first needle 11, through the biofuel cell 3 and finally out of the second needle 12, and
an inactive condition wherein it does not allow biological fluid to pass through the biofuel cell 3.

The pump 4 may also include at least one electric motor, optionally supplied with DC voltage (optionally at a voltage corresponding to the voltage deliverable by the battery 6 essentially equal to 3.3 Volt). The control unit 50 is active in command on the electric motor to control the activation/inactive condition of the pump.

As mentioned, the medical device 1 may comprise the control unit 50 carried by the holder 2, in particular the top layer 2a, and placed in the inner volume of the case; the control unit 50 is configured for operating in command over one or more of the above components. The control unit 50 may include a digital microprocessor that can be powered by the battery 6 and a memory for storing data, such as the threshold value of the control parameter, the electrical threshold value, and the threshold value of minimum charge of the battery 6. The memory may be a non-rewritable type, such as a ROM memory wherein predefined data are stored, or the memory may be a rewritable memory, such as a solid-state type, wherein editable values may be stored, such as by means of a data-entry unit (not shown in the accompanying figures).

The control unit 50 is connected to the sensor 5 and activate in control on the pump 4, which is configured to perform an electricity production procedure for charging the battery 6 during which the control unit 50 commands the activation of the pump 4 to produce electricity to be supplied to the battery 6. In detail, the power production procedure may include the following steps:
receiving one or more signals from the sensor 5, representative of the control parameter of the biological fluid, e.g., blood and/or interstitial fluid, of the patient,
processing each signal emitted by the sensor 5 to estimate respective values of the control parameter,
comparing each estimated value of the control parameter with a threshold value, and
based on said comparison, commanding the activation of pump 4 for supplying a predetermined amount of the biological fluid of the patient, e.g., blood and/or interstitial fluid, to the biofuel cell 3 for power generation.

In detail, the control parameter may include the glycemic rate in the blood of the patient. In this condition, the comparison step of the power generation procedure involves comparing the estimated value of glucose in the blood of the patient with the threshold value (optionally comprised between 80 mg/dl and 100 mg/dl). The control unit 50 is then configured for commanding the activation of the pump 4 in the event that the estimated glucose value exceeds the aforementioned range so that the oxidation of glucose allows the biofuel cell 3 to produce electricity to be supplied to the battery for recharging and, at the same time, for reducing the glycemic rate in the blood of the patient. During the activated condition of the pump 4, the control unit 50 is configured for continuously receiving signals from the sensor 5, representative of the control parameter for continuous estimation of the glucose value in the fluid of the patient; each estimated glucose value is compared with the threshold value so that the control unit may keep the pump 4 in the activated condition only when the glucose value is higher than the threshold value.

For example, the control unit 50, once the presence of a glucose value in the fluid of the patient above the threshold value has been established, may be configured for calculating and treating an amount of fluid sufficient to reestablish the values of glucose of the fluid at or below the threshold value. The control unit 50 is then configured for commanding the activation of the pump 4 for a predetermined time interval wherein it supplies the previously calculated amount of fluid to the biofuel cell 3 for generating electric energy. The amount of fluid may be a function of a flow rate of fluid deliverable by the pump 4: thus, the medical device 1 may include a flow rate sensor connected to the control unit 50 and configured for emitting a signal representative of a flow rate of fluid of the patient passing through the pump 4. The power generation procedure may further comprise the steps of receiving the signal emitted by the flow rate sensor, based on said signal estimating the flow rate of fluid being delivered by the pump 4, and consequently commanding the activation of the pump 4 for the predetermined period of time during which the amount of fluid of the patient is delivered to the biofuel cell 3.

Thus, the control unit 50 allows for using hyperglycemic conditions in the fluid of the patient for the production of electricity via the biofuel cell 3 for the subsequent recharging of the battery 6; in this way, it is possible to recharge the medical device 1 and keep the fluid glycemic rate in the patient under control so as to avoid hyperglycemic conditions. The medical device 1 may include a data transmitter 15 carried by the holder 2, optionally by the rigid layer 2a and housed in case 1a. The data transmitter 15 is configured for transmitting at least one data signal to an external mobile device 120, such as a smartphone, tablet, or computer. The data transmitter 15 is connected to the control unit 50 and configured for commanding the activation of the data transmitter 15, for allowing a monitoring signal to be sent to the external mobile device 120 including at least the values of the control parameter. In particular, the control unit 50 is active in command on data transmitter 15 and is configured for performing a monitoring procedure comprising the following steps:
receiving one or more signals from the sensor 5,
process each signal emitted by the sensor 5 and estimating respective values of the control parameter of the fluid,
comparing each estimated value of the control parameter with a threshold value, and
commanding the activation of the data transmitter 15 to emit a monitoring signal to the external mobile device 120 representative of the values of the control parameter of the fluid of the patient.

Therefore, the monitoring procedure allows the patient to view, via the external mobile device 120, the blood glucose values detected by the sensor 5.

The control unit 50 may also be configured for detecting a malfunctioning condition of the medical device 1, which may be determined, for example, according to the temperature value detected by the temperature sensor in the energy converter 7, for example, if it exceeds a predetermined threshold value. The control unit 50, if it detects the malfunction condition, is configured for commanding the data transmitter 15 to send a respective signal to the external mobile device 120 for notifying the patient of a malfunction of the medical device 1.

The control unit 50 is configured for commanding the data transmitter 15 to issue an alarm signal when it determines a value of the charging level of the battery 6 lower than the threshold value of minimum charge. The control unit 50, through the data transmitter 15, allows the patient to be alerted of a low charging level of the battery. The data transmitter 15 is also configured for notifying the patient if a malfunctioning condition of the medical device 1 is detected, or to send the monitoring signal representative of the blood glucose values detected by the sensor 5. The data transmitter 15 may include a wireless data transmission module, such as one that may take advantage of Bluetooth, Wi-Fi or infrared technology.

In a variant not shown in the accompanying figures, the medical device 1 may be without the energy converter 7 and configured to decrease or modulate the percentage of glucose in the blood of the patient without recharging the battery 6 of the medical device 1 itself. In the latter configuration, the medical device 1 is configured, as previously described, to compare the estimated values of the control parameter with the value of a glucose threshold parameter in the fluid of the patient, for example, comprised between 60 mg/dl and 100 mg/dl. Depending on the outcome of said comparison, the control unit 50 may be configured for commanding the activation of the pump 4 to supply the predetermined amount of fluid of the patient to the biofuel cell 3 and allow oxidation of the glucose in the fluid of the patient.

The medical device 1 may include a charge receiver 16 (FIG. 3) directly connected to the battery 6 or connected to battery 6 via the energy converter 7; the charge receiver 16 is configured to receive energy from a source external to the medical device 1 and generating electrical energy for charging the battery 6. The charge receiver 16 may, for example, comprise an antenna carried by the holder 2, optionally housed in the inner volume of the case, configured for receiving electromagnetic waves, e.g., microwaves, to generate electrical voltage useful for charging the battery 6. For example, the antenna may be a coil or winding suitable for generating an alternating electric current that, when converted to direct current, is used for charging the battery 6. The alternating electric current generated by the antenna may be sent to the energy converter 7 or an AC-DC converter, and then sent to the battery 6.

As shown in FIG. 8, the medical device 1 may also include a charge transmitter 17 connected to the battery 6 and configured for emitting electromagnetic waves employable for charging an additional device unrelated to the medical device 1, such as an implantable device 101 present in the patient. The charge transmitter 17 may include an antenna carried by the holder 2, optionally housed in the inner volume of the case, configured for generating electromagnetic waves, optionally microwaves, for charging the implantable device 101. For example, the antenna may be a coil or winding suitable for generating a magnetic field configured for inducing in the implantable device 101, an alternating electric current usable for charging the implantable device 101 itself. The charge transmitter 17 is connected to the control unit 50 and configured for commanding it, based on a charging level of the battery 6, to emit electromagnetic waves for charging the implantable device 101. As previously mentioned, the control unit 50 is configured for detecting a charging level of the battery 6 and comparing it with the threshold value of minimum charge (e.g., comprised between 20% and 30% of the total storable energy in the battery 6). If the charging level is higher than the threshold value of minimum charge, the control unit 50 is configured for commanding the charge transmitter 17 to emit electromagnetic waves to charge the implantable device 101; vice versa, the control unit 50 is configured for commanding, via the data transmitter 15, to emit an alarm signal to notify the patient of a low charging level of the battery 6. The operation of the medical device in cooperation with the implantable device will be described in detail later in connection with the description of a medical system.

The medical device 1 is employable to treat subjects with diabetes or subjects without any particular disease who wish to keep certain blood parameters, such as glycemic rate, under control. Thus, the medical device 1 is employable to perform therapeutic treatment methods; however, it may also be used by healthy subjects for mere monitoring of blood parameters, without performing any therapeutic treatment.

### Recharging Method of the Medical Device

A method of recharging performed by the medical device 1 above and/or according to the accompanying claims is also described.

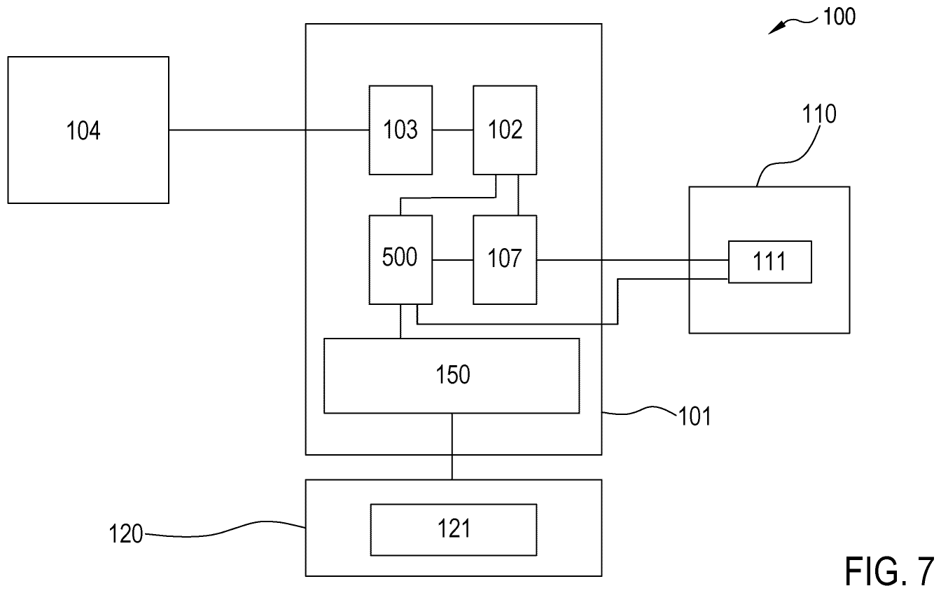

As shown in FIG. 6, the recharging method involves the steps of detecting, using the sensor 5, one or more signals representative of a control parameter of the fluid of the patient (e.g., blood and/or interstitial fluid), and estimating respective values of the control parameter of the fluid (step 200). The method may also include a step of comparing each estimated value of the control parameter with the threshold value, the latter being comprised between 60 mg/dl and 100 mg/dl (step 202). If results from the comparison in step 202 that the estimated value of the control parameter is lower than the threshold value, the method involves detecting additional values of the control parameter using the sensor 5 (see the return line indicated by the reference number 201). If, on the other hand, the comparison in step 202 results in the estimated value of the control parameter being greater than the threshold value, the method involves commanding the activation condition of the pump 4 (step 203) to allow the biological fluid of the patient to pass through the biofuel cell and consequently to allow energy production.

The method also includes a step of estimating a value of an electrical parameter (e.g., a voltage signal output from the biofuel cell) representative of the electrical energy produced by the biofuel cell (step 204) and then, comparing it with an electrical threshold value comprised between 0.2 Volt and 0.4 Volt (step 205). If the electrical parameter value is equal to or greater than the electrical threshold value, the method involves a step of supplying the energy produced by the biofuel cell to the battery 6 to recharge it (step 207). If the value of the electrical parameter is lower than the electrical threshold value, the method involves detecting additional values of the control parameter using the sensor 5 (see the return line indicated by the reference number 206) and interrupting the transit of the voltage signal output from the biofuel cell 3.

The method also involves to cyclically performing the above steps 200-207 until the battery 6 has a sufficiently high state of charge to allow proper power supply to each electrical/electronic element of the medical device.

The method may also involve performing one or more steps to detect and determine the charging level of the battery 6; specifically, the method involves:
detecting a charging level of the battery 6,
comparing the charging level of the battery with a threshold value of minimum charge (e.g., comprised between 20% and 30% of the total storable energy in the battery 6), and
if the charging level is lower than the threshold value of minimum charge, emitting an alarm signal (via the data transmitter 15) to notify the user of a low level of remaining charge of the battery 6.

The method may also include a charging phase of the battery 6 by recurring to the use of an external charging source, such as a wall charger that may be physically connected to the battery 6 via a miniUSB, microUSB, or USB-C connector, or via an inductive charging base.

As mentioned above, the recharging method may be a method that can be performed by healthy subjects for the mere monitoring of parameters in the blood. However, the method may also be a therapeutic treatment method when used by subjects with diabetes and performed for the purpose of maintaining blood glucose levels below predetermined threshold values.

### Implantable Device

An implantable device 101 that may be used to charge an implantable electronic component 110 such as a pacemaker, which may also be installed under the skin of the patient and configured for performing predetermined functions, is also described.

The implantable device 101 may be a module independent from the implantable electronic component 110, which may be installed in the biological environment of the patient, such as in a subclavicular or abdominal area, suitable for interacting with the implantable electronic component itself. The implantable device 101 is electrically and/or communicatively connectable to the implantable electronic component 110, such as by means of an electrical cable.

The implantable device 101 includes a case 101a suitable for housing one or more electrical/electronic components. The case is of the watertight type, suitable for isolating each component from the biological environment of the patient and preventing the passage of fluids or gases such as to jeopardize the proper functioning of the implantable device itself.

As shown in FIG. 8, the implantable device 101 includes a battery 102 housed in the case 101a and configured for powering each of the electrical/electronic elements of the implantable device 101. The battery 102 may be a rechargeable solid-state type battery, optionally lithium iodide, or a conventional lithium-ion type battery. The battery 102 has a charging capacity comprised between 2 Ampere-hour and 10 Ampere-hour and is configured for supplying a continuous output voltage of essentially 3.3 Volt. The battery 102, besides of enabling the electrical/electronic elements of the implantable device 101 to be powered, also allows energy to be stored for charging the implantable electronic component 110 as detailed in the following.

The implantable device 101 may include a control unit 500 connected to the battery 102 and configured to receive a signal representative of a charging level of the same battery 102. The control unit 500 is also configured to estimate a value of the charging level of the battery 102 and compare it with a threshold value of minimum charge, for example, comprised between 20% and 30% of the total storable energy in the battery 102. If the value of the charging level is lower than the threshold value of minimum charge, the control unit 500 is configured to notify the patient of a low charging level of battery 102 and request recharging.

The implantable device 101 may also include a charge receiver 103 connected to the battery 102 of the implantable device 101, configured to receive power from an external power source, such as from the medical device 1 described above or from an external charging source 104, for charging the battery 102. The charge receiver 103 of the implantable device 101 may, for example, be an antenna housed in the case 101a, configured to receive electromagnetic waves, such as microwaves, to generate an electrical voltage. For example, the antenna may be a coil or winding suitable for generating an alternating electric current that, when converted to direct current, is used to charge the battery 102.

The implantable device 101 may also include a charge transmitter 107 housed in the case 101a of the implantable device 101 and configured for allowing charging of implantable electronic component 110. The charging transmitter 107 may be a conventional DC-DC power converter, such as a step-up regulator, buck-boost converter, or Sepic converter, having an input voltage comprised between 0.5 Volt and 5.5 Volt, and a constant output voltage of 3.3 Volt. The implantable device 101 may include a data communication module 150 housed in the case 101a of the implantable device, configured for transmitting data to an external mobile device 120 such as a smartphone, tablet, or computer. The data communication module 150 is connected to the control unit 500 of the implantable device 101, configured to send an alarm signal to the patient to warn the patient itself of a malfunction of the implantable device or a low remaining charge of the battery 102 of the implantable device itself. The control unit 500, if it detects a low charging level of the battery 102 of the implantable device, such as when the value of the charging level of the battery 102 is lower than the threshold value of minimum charge, is configured for commanding the data communication module 150 to emit the alarm signal. The data communication module 150 may include a wireless data transmission module, such as using Bluetooth, Wi-Fi, or infrared technology.

As anticipated, the implantable device 101 may has a control unit 500, housed in the case 101a and connected to battery 102, the charge transmitter 107 and the data communication module 150. The control unit 500 of the implantable device 101 may implement fully analog logic, or include a microprocessor implementing a digital logic. In one variation, the implantable device 101 may be integrated into the implantable electronic component 110.

### Medical System

A medical system 100, comprising an implantable electronic component 110 that may be installed under the skin and of a patient and configured for performing predetermined functions, is also described. In an example, the implantable electronic component 110 may be a pacemaker pre-positioned to electrically stimulate the contraction of the heart of the patient, or a cardiac defibrillator, a sacral nerve stimulator, or a glucose meter. The implantable electronic component 110 may have an electric energy accumulator 111 for power supplying one or more of the electrical/electronic components of the implantable electronic component itself. The electric energy accumulator 111 may, for example, be a rechargeable battery of the solid-state type, optionally lithium iodide type, or a conventional lithium-ion battery.

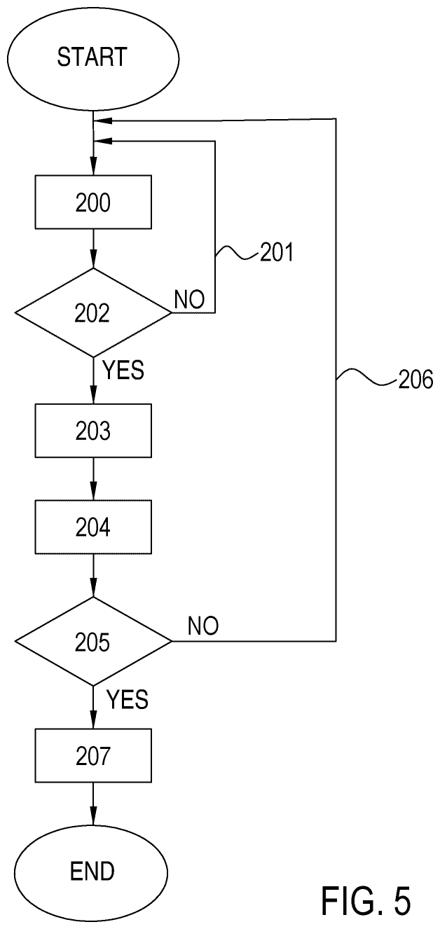

The system may also include the implantable device 101 described above which is suitable for interacting with the implantable electronic component, for example, for allowing its recharging. In the example of FIG. 4, the implantable device 101 is an independent module from the implantable electronic component 110 that may be installed in the biological environment of the patient, such as in a subclavicular or abdominal area, which is suitable for interacting with the implantable electronic component itself. The implantable device 101 is electrically and/or communicatively connected to the implantable electronic component 110, such as by an electrical cable.

The medical system 100 also includes a charging source 104 configured to transmit electrical energy to the charge receiver 103 of the implantable device 101 for recharging of the battery 102 of the implantable device. The charging source 104 includes a transmitting antenna configured for generating electromagnetic waves, optionally microwaves, detectable by the charge receiver 103 for generating electrical energy. For example, the charging source 104 may comprise a wireless charging base, external to the patient. The charging source 104 may, in addition to or as an alternative to the charging base, comprise at least one medical device 1 according to the preceding description. To allow efficient energy transfer, it is preferable to place the charging source 104 in close proximity to the charge receiver 103 of the implantable device 101, particularly at a distance comprised between 5 mm and 30 mm. In case the charging source has one or more electrical windings, these may be superimposed on the electrical windings of the charge receiver 103 of the implantable device 101.

As mentioned above, the implantable device 101 may include a charge transmitter 107 housed in the case 101a of the implantable device 101 and configured for charging the implantable electronic component 110. The charge transmitter 107 is connected to the battery 102 of the implantable device 101 and also connected to the implantable electronic component 110 to recharge the electric energy accumulator 111. The charging transmitter 107 may be a conventional DC-DC power converter, such as a step-up regulator, buck-boost converter or Sepic converter, having an input voltage comprised between 0.5 Volt and 5.5 Volt, and a constant output voltage of 3.3 Volt.

As mentioned above, the implantable device 101 may include a data communication module 150; in an embodiment of the implantable device 101, the data communication module 150 is also configured to communicate with the medical device 1 (when the charging source 104 includes said medical device 1) to send it an alarm signal representative of a low charging level of the battery 102 of the same implantable device. The control unit 50 of the medical device 1 is configured to receive such signal and command its charge transmitter to supply energy to the implantable device for charging the battery 102 of the implantable device. Note that the control unit 50 of the medical device, before commanding to supply energy to the implantable device 101, checks the remaining charging level of the battery 6. In an example, following receipt of the alarm signal from the implantable device, the control unit 50 of the medical device 1, estimates the charging level of its battery 6 and compares it with the threshold value of minimum charge. If the remaining charging level of the battery 6 of the medical device 1 is higher than the threshold value of minimum charge, the control unit 50 of the medical device 1 commands to supply energy via the charge transmitter 17 of the medical device 1 to recharge the energy accumulator 111 of the implantable electronic component 110.

As anticipated, the implantable device 101 may have a control unit 500; the control unit 500 is also connected to the implantable electronic component 110 and in particular to the electric energy accumulator 111, configured to estimate a residual charging level of the same electric energy accumulator 111. The control unit 500 is also configured to compare the estimate of the residual charging level with the threshold value of minimum charge. If a low residual charging level of the energy accumulator is determined, such as if the residual charge estimate is lower than the threshold value of minimum charge, the control unit 500 is configured to command the transfer of energy from the battery 102 to the electric energy accumulator 111 of the implantable electronic component, allowing it to be recharged. The medical system 100 may include a mobile device 120 that may be connected to the implantable device 101 and configured to display data sent by the latter. For example, the mobile device may be a smartphone, tablet, or computer, having a screen 121 for allowing the patient to view the alarm signal sent by the data communication module 150. The mobile device 120 may also be connected to the medical device, configured to receive the alarm signal issued by the medical device itself and notify the patient of a low level of the battery 6 of the medical device.

In a variant shown in FIG. 3, implantable device 101 is integrated into the implantable electronic component 110. In such a configuration, the implantable electronic component 110 comprises a single battery, which replaces the previously mentioned battery 102 and the electric energy accumulator 111, while keeping unchanged the operation modes of the medical system described above.

### Charging Method of the Medical System

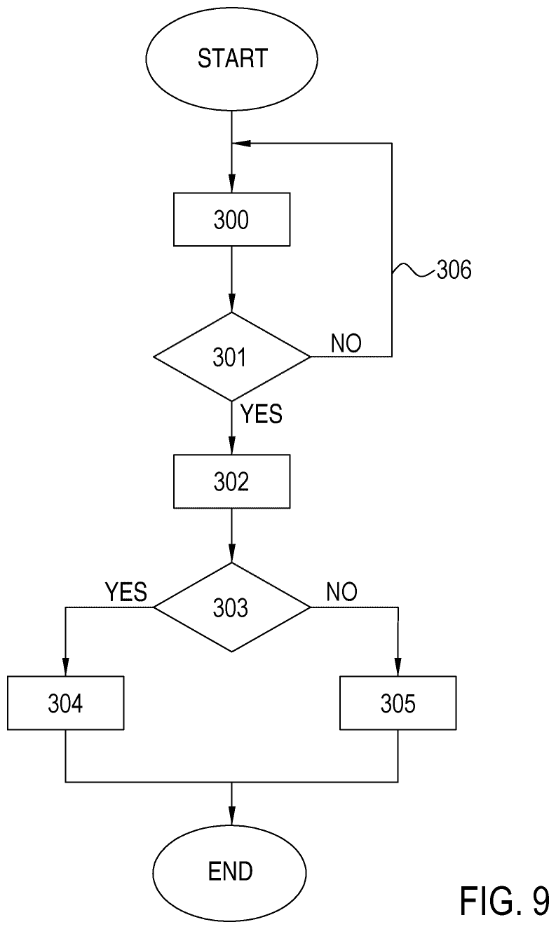

A method for charging the medical system 100 is also described. As shown in FIG. 9, the method involves a first step of estimating one or more values of a residual charging level of the electric energy accumulator 111 of the implantable electronic component (step 300) and comparing each estimated value of the charging level of the electric energy accumulator 111 with a respective threshold value of minimum charge comprised between 20% and 30% of a total accumulable energy of the electric energy accumulator 111 (step 301). In case the above comparison indicates an estimated value of the charging level higher than the threshold value of minimum charge of the energy accumulator, the method involves re-performing step 300 (see recirculation line 306 in FIG. 9). In case the above comparison indicates an estimated value of the charging level lower than the threshold value of minimum charge of the electric energy accumulator 111, the method involves estimating one or more values of the charging levels of the battery 102 of the implantable device (step 302) and comparing them with the threshold value of minimum charge of the battery 102 (step 303). In case the residual charge level of the battery 102 of the implantable device is higher than the threshold value of minimum charge, for example, comprised between 20% and 30% of a total accumulated energy from the battery 102 of the implantable device 101, the method involves a step of commanding the implantable device 101 to supply, via the charge transmitter 107, electrical energy accumulated in the battery 102 to the electrical energy storage 111 (step 304). In case that the residual charging level of the battery 102 is lower than the threshold value of minimum charge, the method involves sending an alarm signal, via the data communication module 150, to the mobile device 120 to notify the patient of a low residual charging level of both the battery 102 of the implantable device 101 and the electric energy accumulator 111 of the implantable electronic component 110 (step 305).

The charging of the electrical energy storage 111 of the implantable electronic component is performed by placing the charging source 104 close to the implantable device 101, for transferring energy between the two devices. This step involves placing the charging source 104 in close proximity to the charge receiver 103 of the implantable device, such as at a distance comprised between 5 mm and 30 mm.

Thus, the method involves a step of receiving and storing energy accumulated by the battery 102 of the implantable device 101 via the charge receiver 103 and transferring energy, via the charge transmitter 107 of the implantable device, to the electric energy accumulator 111 of the implantable electronic component 110. In a variant, if the residual charging levels of the battery 102 of the implantable device 101 and the electric energy accumulator 111 are lower than respective threshold value of minimum charge, the method includes a step of transmitting an alarm signal to the medical device 1 via the data communication module 150. Following receipt the alarm signal via a data receiver of the medical device, the method includes a step of receiving and estimating one or more values of the charging level of the battery 6 of the medical device 1 and comparing them with a threshold value of minimum charge of the medical device 1, for example, comprised between 20% and 30% of a total energy storable by said battery 6 of the medical device 1. In the event that the value of the charging level of the battery 6 is higher than the threshold value of minimum charge, the method includes commanding to the medical device 1, the transfer of energy, via its own charge transmitter 17, from the battery 6 of the medical device 1 to the battery 102 of the implantable device 101. Subsequently, the method involves transferring energy from the battery 102 of the implantable device 101 to the electrical energy storage 111 of the implantable electronic component 110 via the charge transmitter 107 of the implantable device 101.

1-15. (canceled)

16. Medical device comprising:
a holder applicable on the skin of a patient,
at least one rechargeable battery carried by the holder,
at least one biofuel cell carried by the holder and connected to the battery, wherein the biofuel cell is configured for receiving a fluid from the patient and producing electrical energy for recharging the battery,
at least one pump carried by the holder and configured for supplying the fluid of the patient to the biofuel cell,
at least one sensor carried by the holder and configured for emitting a signal representative of a control parameter of the fluid of the patient,
a control unit connected to the pump and the sensor and active in control on said pump, wherein the control unit is configured for performing a power generation procedure comprising the following steps:
receiving a signal from the sensor,
processing the signal emitted by the sensor and estimating respective values of the control parameter of the fluid of the patient,
comparing the estimated value of the control parameter with a threshold value, and
based on the comparison, commanding the activation of the pump for supplying the fluid of the patient to the biofuel cell for producing electricity.

17. The medical device according to claim 16, wherein the control parameter includes at least one of: blood glucose, glycated hemoglobin, oxygen concentration, heart rate, blood pressure, temperature, amino acid or fat concentration.

18. The medical device according to claim 16, wherein the sensor is an optical glucose sensor.

19. The medical device according to claim 16, wherein the sensor includes at least one selected in the group of: an infrared sensor, a luminescence sensor, an electromagnetic radiation sensor, an acoustic sensor, an ultrasonic sensor, an impedance measurement circuit, a radio frequency sensor.

20. The medical device according to claim 16, wherein the control unit, during the estimation step of the value of the control parameter of the power generation procedure, is configured for estimating the value of glucose in the fluid of the patient,
wherein the control unit, during the subsequent comparison step of the power generation procedure, is configured for comparing said fluid glucose value with a threshold value.

21. The medical device according to claim 20, wherein the threshold value is comprised between 60 mg/dl and 100 mg/dl.

22. The medical device according to claim 20, wherein the activation step of the pump of the power generation procedure is performed in case the control unit determines an estimated glucose value greater than the threshold value.

23. The medical device according to claim 16, wherein the control unit is connected to the biofuel cell which is configured for emitting a signal representative of an electrical parameter representative of the electrical energy produced, wherein the power generation procedure, executed by the control unit, also includes the following steps:
receiving the signal from the biofuel cell,
based on the signal emitted by the biofuel cell, estimating a value of the electrical parameter,
comparing the value of the electrical parameter with an electrical threshold value, based on said comparison, commanding the transmission of the electrical energy produced by the biofuel cell to the battery.

24. The medical device according to claim 23, wherein the control unit is configured for commanding the transmission of electrical energy produced by the biofuel cell to the battery when the value of the electrical parameter is greater than the electrical threshold value.

25. The medical device according to claim 23, wherein the electrical parameter includes at least one of: an electrical voltage measured at the ends of the biofuel cell, an electrical current, an electrical energy, an electrical resistance.

26. The medical device according to preceding claim 23, wherein the battery is electrically connected to the pump, the sensor and the control unit, wherein the battery is configured for providing electrical energy to power the pump the sensor and the control unit.

27. The medical device according to claim 16 comprising an energy converter interposed between the battery and the biofuel cell, wherein said energy converter is configured for receiving in input electrical energy produced by the biofuel cell and emitting in output a predetermined electrical energy for charging the battery, wherein the output electrical energy from the energy converter is greater than the input electrical energy.

28. The medical device according to claim 27, wherein the energy converter includes a voltage regulator configured for receiving as input an electric voltage generated by the biofuel cell and emitting in output a predetermined electric voltage for charging the battery, wherein the output electric voltage from the voltage regulator is higher than the input electric voltage.

29. The medical device according to claim 16 comprising a first and second needle suitable for allowing the passage of fluid of the patient,
wherein the first needle is in fluid communication with the pump and configured for drawing fluid from the patient and allow it to be sent to the pump,
wherein the second needle is in fluid communication with the biofuel cell and configured for receiving incoming fluid from the biofuel cell and feeding it back into the patient.

30. The medical device according to claim 16, wherein the first and second needles are micro-needles configured for allowing the passage of blood and/or interstitial fluid of a patient.

31. The medical device according to claim 30, wherein the at least one first needle has a number of micro-needles comprised between 100 and 5000, wherein each micro-needle of said at least one first needle has a length comprised between 100 and 500 μm and a diameter comprised between 5 and 50 mμ,
wherein the at least one second needle has a number of micro-needles comprised between 100 and 5000, wherein each micro-needle of said at least one second needle has a length comprised between 100 and 500μm and a diameter comprised between 5 and 50 μm.

32. The medical device according to claim 30, wherein the holder comprises at least one plaster having at least one adhesive layer defining a contact surface of the holder suitable for directly contacting the skin of a patient, wherein the holder extends in thickness between the contact surface and an opposing holder surface.

33. The medical device according to claim 30, wherein the holder directly carries the micro-needles of said first and second needles, wherein the micro-needles of said at least one first needle are distributed over the contact surface of the holder with a density greater comprised between 50 and 1000 micro-needles per cm²,
wherein the micro-needles of said at least one second needle are distributed over the contact surface of the holder with a density comprised between 50 and 1000 micro-needles per cm².

34. The medical device according to claim 16 comprising a charge receiver connected to the battery and configured for receiving electromagnetic waves from an external charger and generating electrical energy, due to said electromagnetic waves, wherein the charge receiver is configured for transmitting electrical energy to the battery to recharge it.
